(19) United States
(12) Patent Application Publication
(10) Pub. No.: US 2009/0274662 A1
(43) Pub. Date: Nov. 5, 2009

(54) Methods And Kits For The Treatment Of Inflammatory Bowel Disorder Conditions

(76) Inventors: Simon Henry Magowan, Loveland, OH (US); Bruce Rudolf Yacyshyn, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/433,909

(22) Filed: May 1, 2009

Related U.S. Application Data
(60) Provisional application No. 61/049,450, filed on May 1, 2008.

(51) Int. Cl. A61K 45/00; A61P 1/14

(52) U.S. Cl. 424/93.4

(57) ABSTRACT

The present invention comprises methods and kits that are useful for the treatment an inflammatory bowel disease. The methods comprise administering to a mammal in need of treatment a composition comprising an active agent in combination with a probiotic and/or optionally an antibiotic.

### CROSS REFERENCE TO RELATED APPLICATIONS

This application claims the benefit of U.S. Provisional Application No. 61/049,450 filed on May 1, 2008.

### FIELD OF THE INVENTION

The present invention is directed to methods and kits that are useful for the treatment an inflammatory bowel disorder. The methods comprise administering to a mammal in need of treatment a composition comprising an active agent, in combination with a probiotic and/or optionally an antibiotic.

### BACKGROUND OF THE INVENTION

The large bowel in humans and to a lesser extent the small bowel, contain large concentrations of various enteric bacteria. Generally, patients will have no pain, cramping, diarrhea, or constipation if the bacterial contents are not infected with pathogenic strains which may colonize the bowel and remain there for prolonged periods of time. Acute infections and some chronic infections of the bowel flora can however cause inflammatory changes in the lining and in the deeper layers of the colonic wall. When inflammation is visible in the deeper layer of bowel wall and is associated with irritable bowel symptoms, the condition is called inflammatory bowel disorder and includes such diseases as inflammatory irritable bowel syndrome and microscopic colitis. These diseases are conditions recognized and known to arise from an inflammatory condition of the colon. In particular, inflammatory irritable bowel syndrome and microscopic colitis are known to be caused by a precipitating infection by a pathogenic organism or organisms. The degree of inflammation in inflammatory irritable bowel syndrome and microscopic colitis is much less than inflammatory bowel diseases such as ulcerative colitis and/or Crohn's disease.

Normally, there are no visible colonoscopic abnormalities in inflammatory bowel disorders that include inflammatory irritable bowel syndrome and/or microscopic colitis. Instead, inflammatory bowel disorders are characterized by inflammation on histology. Inflammatory irritable bowel syndrome and microscopic colitis show evidence of inflammation on histology obtained by colonoscopy and the histology shows an increase in inflammatory cells. Inflammatory irritable bowel syndrome and microscopic colitis can be referred to as specific bowel disorders because there are specific diagnostic criteria such as inflammation in the wall of the colon that can help diagnose it. Stool samples can be collected and the stool tests are positive for inflammatory markers of inflammation, and the patient complains of symptoms referable to the colon, such as urgency, diarrhea, flatulence, cramping, the diagnosis of inflammatory irritable bowel syndrome or microscopic colitis can also be made. Collectively, between 5% and 25% of the western population in different age groups may suffer from these disorders which have also been termed spastic colon, unstable colonic neurosis, spastic colitis, or mucous colitis. In a classic case there is a triad of symptoms including low abdominal pain relieved by defecation, alternating constipation/diarrhea and the passage of small caliber stools. In some patients there may be accompanying watery diarrhea with or without pain. Distension, flatulence, wind and at times nausea and headaches may also be accompanying systemic symptoms. Some patients also experience at times diarrhea alternating with constipation.

To date, there is no therapy that has addressed the inflammatory pathogenesis of inflammatory irritable bowel syndrome. Conventional treatments for Irritable Bowel Syndrome (IBS) have been unsatisfactory as exemplified by the large number of therapies that have from time to time been recommended or studied. These have included psychotherapy, dietary regimens, anti-spasmodic agents, anti-cholinergics, antidepressants, bulking agents, various receptor antagonists, carminatives, opiates, and tranquillizers, all without substantial success. Indeed, there is no evidence that a cure is possible. Yet IBS is one of the most common of all the gastrointestinal illnesses and though not life threatening causes great distress especially to those severely affected, and may bring a feeling of frustration and helplessness, being generally life long. In particular, diarrhea-predominant IBS can cause incontinence in some patients and, for example, the inability of being sure that one can reach ones employment causing some to drive from rest room to rest room on their way to work. In some patients urgency is so severe that they can only hold their motions for a few seconds.

The treatment or prophylaxis of inflammatory irritable bowel syndrome may be provided per the current application with an effective amount of aminosalicylate, sulfasalazine, 5-aminosalicylic, 4-aminosalicylic acid, benzalazine, dihydrochloride salt, olsalazine, balsalazide, bismuth subsalicylate, and mixtures thereof. Additionally, a therapeutic manipulation of the colonic flora may decrease colonic inflammation and ameliorate symptoms.

### SUMMARY OF THE INVENTION

The present invention is directed to methods and kits for the treatment of inflammatory bowel disorder. In one embodiment, the invention is directed to a method of treating an inflammatory bowel disorder; comprising administering to a mammal in need of such treatment a composition comprising at least one active agent and a probiotic. In another embodiment, the invention is directed to a method of treating an inflammatory bowel disorder, the method comprising administering to a mammal in need of such treatment an active agent, a probiotic, and optionally an antibiotic.

In yet another embodiment, the invention is directed to kits, wherein the kits comprise an active agent which can be aminosalicylate, sulfasalazine, 5-aminosalicylic acid, 4-aminosalicylic acid, benzalazine, dihydrochloride salt, olsalazine, balsalazide, bismuth subsalicylate, and mixtures thereof, and a probiotic, which may be a Bifidobacterium sp. but need not be. The kits may optionally comprise an antibiotic.

### DETAILED DESCRIPTION OF THE INVENTION

The present invention comprises methods and kits that are useful for the treatment an inflammatory bowel disorder.

As used herein, the term “administration”, “administering”, “or the like with respect to the user means that the user is administered, is directed to administer or, with reference specifically to “oral administration,” or “orally administering,” ingests or is directed to ingest, the composition. For example, the administration may be oral administration, parenteral administration, topical administration, buccal administration, rectal administration, or the like, or any combination thereof. As but one example, the active agent may be administered orally or rectally, while the probiotic may be administered orally. In one embodiment, all components are administered through oral administration.

As used herein, the term “inflammatory bowel disorder”, includes a disorder that is diagnosed by at least one of the following criteria 1) inflammation in the wall of the colon and/ or 2) a stool sample that tests positive for inflammatory markers of inflammation in combination with a patient that complains of at least one symptom. The symptoms include but are not limited to low abdominal pain relieved by defecation, alternating constipation/diarrhea, passage of small caliber stools, cramping, diarrhea, constipation, urgency, flatulence, watery diarrhea with or without pain, distension, nausea and or incontinence. Inflammatory bowel disorder includes inflammatory irritable bowel syndrome, irritable bowel syndrome-diarrhea, irritable bowel syndrome-constipation, irritable bowel syndrome-mixed, irritable bowel syndrome-alternating, dyspepsia, microscopic colitis, lymphocytic colitis, collagenous colitis, indeterminant colitis, celiac disease, and combinations thereof.

Except where specific examples of actual measured values are presented, numerical values referred to herein should be considered to be qualified by the word “about”.

All weights, measurements and concentrations herein are measured at 25° C. on the composition in its entirety, unless otherwise specified.

These and other limitations of the compositions and methods of the present invention, as well as many of the optional ingredients suitable for use herein, are described in detail hereinafter.

All percentages, parts and ratios as used herein are by weight of the total composition, unless otherwise specified. All such weights as they pertain to listed ingredients are based on the active level and, therefore do not include solvents or by-products that may be included in commercially available materials, unless otherwise specified.

The composition and methods of the present invention can comprise, consist of, or consist essentially of, the essential elements and limitations of the invention described herein, as well as any additional or optional ingredients, components, or limitations described herein or otherwise useful in compositions intended for use by a mammal, preferably human use.

### Methods of the Present Invention

The present invention is directed to methods and kits for the treatment of an inflammatory bowel disorder.

In one embodiment, the invention is directed to a method of treating inflammatory bowel disorder selected from inflammatory irritable bowel syndrome, irritable bowel syndrome-diarrhea, irritable bowel syndrome-constipation, irritable bowel syndrome-mixed, irritable bowel syndrome-alternating, dyspepsia, microscopic colitis, lymphocytic colitis, collagenous colitis, indeterminant colitis, celiac disease, and combinations thereof, the method comprising administering to a mammal in need of such treatment a composition comprising at least one active agent. In another embodiment, the invention is directed to a method of treating inflammatory bowel disorder selected from inflammatory irritable bowel syndrome, irritable bowel syndrome-diarrhea, irritable bowel syndrome-constipation, irritable bowel syndrome-mixed, irritable bowel syndrome-alternating, dyspepsia, microscopic colitis, lymphocytic colitis, collagenous colitis, indeterminant colitis, celiac disease, and combinations thereof, the method comprising administering to a mammal in need of such treatment an active agent, a probiotic, and optionally an antibiotic.

Inflammatory bowel disorder may be associated with a variety of symptoms including, for example, in a classic case a triad of symptoms including low abdominal pain relieved by defecation, alternating constipation/diarrhea and the passage of small caliber stools. In some patients there may be accompanying watery diarrhea with or without pain. Distension, flatulence, wind and at times nausea and headaches may also be accompanying systemic symptoms. Some patients also experience at times diarrhea alternating with constipation. The methods herein encompass treatment of any of this variety of symptoms.

Additionally, inflammatory bowel disorders are characterized by inflammation on histology. Inflammatory irritable bowel syndrome and microscopic colitis show evidence of inflammation on histology obtained by colonoscopy and the histology shows an increase in inflammatory cells. Stool samples can also be collected and the stool tests are positive for inflammatory markers of inflammation, and the patient complains of symptoms referable to the colon, such as urgency, diarrhea, flatulence, cramping, the diagnosis of inflammatory irritable bowel syndrome or microscopic colitis can also be made.

Wherein the user is directed to administer the composition or component, such direction may be that which instructs and / or informs the user that use of the composition or component (as applicable) may and/or will provide one or more general health and / or general physiological benefits associated with the health care product. For example, such direction may be oral direction (e.g., through oral instruction from, for example, a physician, health professional, sales professional or organization, and/or radio or television media (e.g., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or containing devices associated with the composition (e.g., a label present on a containing device containing the composition). See e.g., the kits described herein.

### Active Agent

The methods of the invention comprise administration of at least one active agent. Nonlimiting examples of the active agent may include those selected from aminosalicylate, sulfasalazine, 5-aminosalicylic acid, 4-aminosalicylic acid, benzalazine, dihydrochloride salt, olsalazine, balsalazide, bismuth subsalicylate, and mixtures thereof.

In one embodiment herein, the active agent may be an aminosalicylate. As used herein, “aminosalicylate” refers to a class of compounds capable of releasing 5-amino-2-hydroxybenzoate or 5-amino-2-hydroxybenzoic acid as an active moiety in vivo. Non-limiting examples include mesalamine (5-amino-2-hydroxybenzoic acid), olsalazine (3,3′-dicarboxy-4,4′-dihydroxyazobenzene), balsalazide ((E)-5-[[4-[[(2-carboxyethyl)amino]carbonyl]phenyl]azo]-2-hydroxybenzoic acid), and sulfasalazine (2-hydroxy-5-[[4-[(2-pyridinylamino)sulfonyl]phenyl]azo]-benzoic acid).

A composition comprising an aminosalicylate may have one or greater than one aminosalicylate in addition to other possible components. The active moiety is illustrated below:

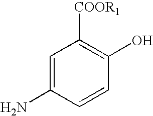

wherein R₁ can be hydrogen or a physiologically relevant counterion and the nitrogen can be further protonated and carry a positive charge along with a physiologically relevant counterion.

Although the examples provided describe the free acid or free amine forms, the term is not so limited and should be interpreted to include the free acid forms, the free amine forms, and any salts thereof. For example, the term “mesalamine” covers the free acid, the free amine, and any salts of mesalamine. The term “mesalamine” is commonly used interchangeably in the art with “mesalazine”, “5-ASA” or “5-aminosalicylic acid”.

In one embodiment herein, the active agent is formulated for release in the small intestines and/or the large intestine, such as for example at the distal portion of the small intestine and in the large intestine. Various illustrative commercial products are suitable for such release including, for example, ASACOL, PENTASA, and LIALDA. Various delayed or sustained delivery technologies are well known for this purpose and need not be described herein.

In accordance with the methods herein, as an example, from about 1 mg to about 8000 mg of the anti-inflammatory may be administered, or from about 50 mg to about 7000 mg, or from about 100 mg to about 6000 mg, or from about 200 mg to about 4800 mg, or from about 1000 mg to about 4800 mg, or from about 1500 to about 4800 mg or from about 2400 mg to about 4800 mg. These doses are typically daily doses, although the ordinarily skilled artisan may manipulate dosing as needed or desired. For example, wherein the anti-inflammatory is available in discrete compositions each comprising 400 mg of the anti-inflammatory, a daily dose of 2400 mg may be administered through administration of six of these discrete compositions per day.

In certain embodiments herein, the active agent is administered prior to administration of the probiotic. In this embodiment, the active agent is administered for a definite period of time (for illustration, three times daily for a ten week period of time); upon conclusion of this illustrative ten week period of time, administration of the probiotic commences and continues for a definite period of time or, optionally, indefinitely.

In other embodiments herein, the active agent is administered contemporaneously with administration of the probiotic. As used herein, “contemporaneously” means that, for any given day of administration, the active agent and the probiotic are both administered on that day (whether at the same time, or at different times during that day).

In other embodiments herein, the active agent is administered prior to administration of the probiotic and is also administered contemporaneously with the probiotic. To illustrate this embodiment, the active agent is administered for a definite period of time (for illustration, three times daily for a ten week period of time); upon conclusion of this illustrative ten week period of time, administration of the active agent continues along with commencement of administration of the probiotic, with active agent and probiotic dosing for a definite period of time or, optionally, indefinitely.

### Probiotic

In certain embodiments, the methods of the present invention may utilize a probiotic. Probiotics have been shown to inhibit pathogen adherence to colonic mucosa, increase immunoglobulin-A (IgA) secretion in Peyer's patches, increase immune activity inhibiting the release of anti-inflammatory cytokines and inhibiting pro-inflammatory cytokines.¹ Probiotics may also interfere with pathogen metabolism.

Advancing age may be associated with decreased bifidobacteria and increased Bacteroides species. It should also be noted that low fiber diets may actually alter the colonic microecology: it has been demonstrated in humans that wheat bran adversely changes the anerobic/aerobic bacterial ratios.² This has implications for the application of probiotics as prophylaxis against bacteroides overgrowth and infection leading to inflammatory bowel disorder.

The probiotic may be, for example, lactic acid bacteria. Non-limiting examples of lactic acid bacteria suitable for use herein include strains of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Bifidobacterium longum, Bifidobacterium infantis, Bifidobacterium bifidum, and Pediococcus cerevisiae, or mixtures thereof. In one embodiment the probiotic is Lactobacillus salivarius, Bifidobacterium infantis, or mixtures thereof. In another embodiment, the probiotic is Bifidobacterium infantis.

As a non-limiting example, strains of Lactobacillus salivarius isolated from resected and washed human gastrointestinal tract as described in WO 98/35014 are preferred. More preferred are the Lactobacillus salivarius strains that are designated UCC 1 and UCC 118, described as being deposited at the National Collections of Industrial and Marine Bacteria Ltd (NCIMB) on November 27, 1996, and accorded the accession numbers NCIMB 40830 and 40829, respectively.

As another non-limiting example, the probiotic is a Bifidobacterium sp. For example, strains of Bifidobacterium sp. isolated from resected and washed human gastrointestinal tract as disclosed in WO 00/42168 may be used herein. One example is the Bifidobacterium infantis strain designated as UCC35624, described as being deposited at the National Collections of Industrial and Marine Bacteria Ltd (NCIMB) on Jan. 13, 1999, and accorded the accession number NCIMB 41003.

The compositions used herein may be given to an individual as part of a dose regimen which may be dependent upon the dosing format used in which the probiotic is incorporated. The composition can comprise at least one strain of a probiotic, alternatively the composition can comprise two strains of a probiotic that are the same or they can be different strains, alternatively the composition can comprise three strains of a probiotic. A single dose of the probiotic comprises at least about 10⁵ cfu of the probiotic. For example, the unit dose provides the mammal being treated with probiotic at a level of from about 1×10⁵ colony forming units (cfu) per dose to about 1×10¹⁵ cfu per dose, alternatively from about 1×10⁷ cfu to about 1×10¹⁴ cfu per dose, alternatively a single dose of the probiotic comprises from about 10⁸ cfu to about 10¹² cfu per dose. As another example, the dose regimen may commence at a higher dose, followed by a lower maintenance dose. See, for example, U.S. application Ser. No. 12/056,702, filed Mar. 27, 2008.

Any of a variety of different dose forms may be appropriate for administration. For example, for oral administration, the unit dose, when provided as a capsule, tablet, or other typical oral dosage form may be swallowed directly. As another example, when provided as a sachet filled with the probiotic, the probiotic may be ingested directly, or mixed with milk, yogurt, or another liquid carrier material. Typically, as an example, a dose form such as a capsules may provide lower dosing amounts than sachets, as the size of the capsule, and its relative easy of ingestion, will limit the amount of the probiotic that can be filled therein.

### Antibiotic

Current standard therapy using antibiotics alone may address acute attacks, but not thereafter the attack (e.g., symptoms may persist). As an example, in order to conform with the current standard of care, in certain embodiments, the methods and kits may optionally comprise administration of an antibiotic.

Antibiotics are commonly known and are selected by one of ordinary skill in the art. To illustrate, the antibiotic may be selected from the group consisting of metronidazole, cephalexin, ciprofloxacin, levofloxacin, moxifloxacin, gemifloxacin, balofloxacin, gatifloxacin, grepafloxacin, pazufloxacin, sparfloxacin, temafloxacin, enoxacin, fleroxacin, lomefloxacin, nadifloxacin, norfloxacin, ofloxacin, pefloxacin, rufloxacin, trovofloxacin, tosufloxacin, clindamycin, tetracycline, chloramphenicol, cefoxitin, cefmetazole, cefotetan, doxycycline, erythromycin, imipenem, meropenem, ticarcillin, pipercillin, mezocillin, tazobactam, ampicillin, and combinations thereof.

As illustrative guidelines, subjects presenting with inflammatory bowel disorder symptoms may be dosed a single type of antibiotic, while those presenting with more severe symptoms may be dosed a combination of antibiotics, including two, three, or even more distinct antibiotics. For example, two distinct antibiotics may be prescribed for those with moderate symptoms, while triple therapy may be prescribed for those presenting with severe symptoms. In many of these moderate to severe cases, intravenous therapy may be appropriate, while typically oral dosing is also appropriate.

Dose level and frequency will be commonly understood in the art, and may be dependent upon the antibiotic employed. Merely as illustration, the methods and kits herein may optionally utilize a daily dose of from about 50 mg to about 6000 mg of antibiotic, or from about 100 mg to about 2500 mg of antibiotic, or from about 250 mg to about 2000 mg of antibiotic. Daily dosing may be administered as a single dose, or divided into multiple doses such as twice daily, three times daily, or four times daily dosing.

To illustrate, the methods and kits herein may utilize administration of metronidazole (for example, FLAGYL). The metronidazole may optionally be administered orally in tablet form, optionally in immediate or sustained release forms. Other useful forms may include topical or intravenous forms, or any other form that would be useful herein. A commonly used oral dose may include administration of from about 250 mg to about 750 mg of the metronidazole on a daily basis until antibiotic administration is complete.

As yet another illustration, the methods and kits herein may utilize administration of cephalexin (for example, KEFLEX, KEFTABS, or BIOCEF). The cephalexin may optionally be administered orally in tablet form, optionally in immediate or sustained release forms. Other useful forms may include powders for suspension, or any other form that would be useful herein. A commonly used oral dose may include administration of from about 250 mg to about 750 mg of the cephalexin on a daily basis until antibiotic administration is complete.

As yet another illustration, the methods and kits herein may utilize administration of doxycycline (for example, VIBRAMYCIN). The doxycycline may optionally be administered orally in tablet form, optionally in immediate or sustained release forms. Other useful forms may include suspensions, or any other form that would be useful herein. A commonly used oral dose may include administration of from about 50 mg to about 300 mg of the doxycycline on a daily basis until antibiotic administration is complete.

As yet another illustration, two or more discrete antibiotics may be utilized. For example, one may choose an antibiotic having anaerobic bacteria kill, and a different antibiotic having aerobic bacteria kill. For example, the methods and kits could utilize ciprofloxacin at a range of from about 250 mg per day to about 2000 mg per day along with metranidazole at a range of from about 250 mg per day to about 6000 mg per day.

In one embodiment, administration of the antibiotic is prior to the administration of the probiotic, prior to the administration of the active agent, or prior to the administration of the probiotic and the active agent. In certain embodiments, administration of the antibiotic is prior to the administration of the active agent and the probiotic. For example, the antibiotic may be administered for a period of from 1 day to about 30 days following an acute attack of a inflammatory bowel disorder, or from 1 day to about 14 days, or from about 7 days to about 10 days. The active agent and the probiotic may also be administered, either contemporaneously or during different time periods.

The foregoing described dosage levels for active agent, probiotic, and antibiotic are based on typical human subjects (e.g., about a 55 to 65 kg subject). Wherein the present composition is used in other mammals, it may be necessary to modify the dosage. Modification of dosage based on the needs of the subject is well within the skill of the ordinary artisan. It is therefore understood that these dosage ranges are by way of example only, and that administration can be adjusted depending on various factors.

The specific dosage of the active agent, probiotic, and antibiotic, as well as the duration of treatment may be interdependent. The dosage and treatment regimen may also depend upon such factors as the specific active agent, probiotic, and antibiotic used, as applicable, the treatment indication, the efficacy of the agent used, the personal attributes of the subject (such as, for example, weight, age, gender, and medical condition of the subject), and compliance with the treatment regimen.

### Kits

In yet another embodiment, the invention is directed to kits, wherein the kits comprise an active agent and a probiotic, which may be a Bifidobacterium sp. but need not be. The kits may optionally comprise an antibiotic.

The active agents, probiotics, and antibiotics, including various embodiments or selections thereof, are as described herein.

In one embodiment, the kits comprise one or more discrete compositions comprising the active agent and one or more discrete compositions comprising the probiotic. For example, the kit may comprise a weekly, monthly, or other periodic dose of the active agent and the probiotic. As an illustrative example, a kit comprising a weekly dose may comprise 7 discrete compositions comprising the probiotic (7 daily doses) and 42 discrete compositions comprising the active agent (7 daily doses, each complete daily dose comprised of six discrete compositions). As another example, a kit comprising a monthly dose may comprise 30 discrete compositions comprising the probiotic (30 daily doses) and 180 discrete compositions comprising the active agent. These kits may optionally comprise the antibiotic, for example in accordance with the number of doses of antibiotic intended for use. For example, wherein it is desired that 10 daily doses of antibiotic is administered prior to administration of the active agent and prior to the probiotic, the kit may optionally contain 10 discrete compositions comprising the antibiotic. Any of a variety of combinations of types and numbers of discrete compositions will be selected by those of ordinary skill in the art.

In certain embodiments, the kits are configured to facilitate dosing compliance. For example, the kits may be particularly advantageous for the purpose of ensuring that the subject is receiving administration of all components (for example, probiotic, active agent, and antibiotic) on the appropriately prescribed schedule. Blister cards or other containing devices appropriately configured may be particularly suitable for clearly illustrating sequence or timing of administration of the various components. Various configurations will be well known to the ordinarily skilled artisan in view of the present specification.

The kits of the present invention may comprise one or more of the active agent, one or more of the probiotic, and antibiotic, optionally together with information which informs a user of the kit, by words, pictures, and/or the like, that use of the kit will provide one or more general health and/or general physiological benefits including, but not limited to, gastrointestinal health benefits (for example relief from, prevention of, treatment of and/or inhibition of a inflammatory bowel disorder), and/or general active agent benefits. Such information need not utilize the actual words used herein, for example, “inflammatory bowel disorder”, “IBD”, “IBS”, Inflammatory IBS“, disease”, “condition”, or “gastrointestinal”, but rather use of words, pictures, symbols, and the like conveying the same or similar meaning are contemplated within the scope of this invention.

In one embodiment, the information is printed on a container holding the composition, e.g., a box, card (e.g., a blister card), or other containing device. These preferred kits may be in the form of one containing device containing the composition, or may be obtained as a plurality of devices each containing the composition. For example, the kits may be obtained as one card, or cases of four, six, seven (e.g., a weekly supply), or eight cards co-packaged together. Additionally, monthly or other types of kits may be obtained.

### Additional Components

The compositions of the present invention can optionally comprise one or more additional components. By way of non-limiting example, an additional probiotic strain; one or more of prebiotics and/or fiber; vitamins; minerals, metals and elements; plant-derived components; fungal-derived components; carotenoids; anti-oxidants; and mixtures/combinations thereof can be used.

The compositions of the present invention can comprise, by way of non-limiting example, one or more probiotic strains of bacteria plus one or more of an additional probiotic strain of bacteria, a prebiotic, a fiber, vitamins, minerals, elements, plant-derived components, fungal-derived components, carotenoids, and antioxidants. Non-limiting examples of some additional components are provided below.

### Prebiotics/Fiber

The compositions can comprise a prebiotic and/or a fiber. As used herein, the term “prebiotic” includes substances or compounds that beneficially affect the host mammal by selectively promoting the growth and/or activity of one or more probiotic bacteria in the gastro-intestinal tract of the host mammal, thus maintaining normal health or improving health of the host. Typically, prebiotics are carbohydrates, (such as oligosaccharides), but the term “prebiotic” as used herein does not preclude non-carbohydrates. Many forms of “fiber” exhibit some level of prebiotic effect. Thus, there is considerable overlap between substances that can be classified as “prebiotics” and those that can be classified as “fibers”.

Non-limiting examples of prebiotics suitable for use in the compositions and methods include psyllium, fructo-oligosaccharides, inulin, oligofructose, galacto-oligosaccharides, isomalto-oligosaccharides xylo-oligosaccharides, soy-oligosaccharides, gluco-oligosaccharides, mannan-oligosaccharides, arabinogalactan, arabinxylan, lactosucrose, gluconannan, lactulose, polydextrose, oligodextran, gentioligosaccharide, pectic oligosaccharide, xanthan gum, gum arabic, hemicellulose, resistant starch and its derivatives, and mixtures and/or combinations thereof.

When present, the compositions can comprise from about 100 mg to about 100 g, alternatively from about 500 mg to about 50 g, and alternatively from about 1 g to about 40 g, of prebiotic, per daily dose of the composition.

### Fiber

As used herein, the term “fiber” means carbohydrate polymers including those naturally occurring in food as consumed, those having been obtained from food raw material by physical, enzymatic or chemical means, and synthetic carbohydrate polymers, which are resistant to digestion and absorption in the small intestine and have partial fermentation in the large intestine.

Non-limiting examples of fiber and analogous carbohydrate polymers suitable for use in the compositions and methods of the present invention include pectins, psyllium, guar gum, xanthan gum, alginates, gum arabic, fructo-oligosaccharides, inulin, agar, beta-glucans, chitins, dextrins, lignin, celluloses, non-starch polysaccharides, carrageenan, and mixtures and/or combinations thereof. In one embodiment, the fiber is glucose polymers, preferably those which have branched chains. Among such suitable fibers is one marketed under the tradename “Fibersol2”, commercially available from Matsutani Chemical Industry Co., Itami City, Hyogo, Japan. Other non-limiting examples of suitable fibers include oligosaccharides, such as inulin and its hydrolysis products commonly known as fructo-oligosaccharides, galacto-oligosaccharides, xylo-oligosaccharides, and oligo derivatives of starch.

The fiber can be provided in any suitable form. A non-limiting example is in the form of a plant material which contains the fiber. Non-limiting examples of suitable plant materials include asparagus, artichoke, onion, wheat, chicory, beet pulp, residues of these plant materials, and mixtures thereof.

When present, the compositions can comprise from about 100 mg to about 100 g, alternatively from about 500 mg to about 50 g, alternatively from about 1 g to about 40 g, of fiber, per daily dose of the composition.

### Vitamins

The compositions of the present invention can optionally comprise one or more vitamins. When certain vitamins, minerals, metals, elements and the like are included as additional components in capsule, tablet and powder forms. The total amount of vitamin(s), by weight, if provided in a premix on or in a suitable carrier, can comprise from about 1% to about 50%, alternatively from about 1% to about 40%, and alternatively from about 2% to about 30%, by weight of the composition. Therefore, when vitamins, minerals, metals and elements are exemplified herein as a % by weight of a composition, such weight % includes any carrier present. With respect to the weight percent of a given vitamin as a percent of a premix or vitamin-carrier mix, such percentages can vary greatly depending on the vitamin and the amount of vitamin desired, as would be understood by one of skill in the art. Generally, however, for vitamins in or on a carrier, the vitamin can comprise, as a weight percent of vitamin to carrier, from about 0.0001% to about 50%, alternatively from about 0.001% to about 45%, alternatively from about 0.001% to about 40%, by weight of the vitamin-carrier composition.

### Vitamin D

The compositions of the present invention can comprise Vitamin D. Non-limiting examples of vitamin D suitable for use in the present invention include vitamin D₃ (cholecalciferol), vitamin D₂ (ergocalciferol) and combinations thereof Additional non-limiting examples include metabolites of vitamin D including calcidiol, calcitriol and combinations thereof. The vitamin D can be derived from natural or synthetic sources, including from an extract of solanum glaucophylum (malacoxylon), trisetum flavescens (goldhafer) or cestrum diurnum. Both the pure vitamin D and/or glycosides of the vitamin D can be used.

Vitamin D is a unique nutrient in that its principal source is not the diet, but via synthesis in the skin upon exposure to UV light, typically sunlight in the summer months. In the skin, 7-dehydrocholesterol, derived from cholesterol, is converted by the action of UV light into Previtamin D₃ (precalciferol), which then undergoes a thermal conversion to Vitamin D₃ (cholecalciferol). Whether the cholecalciferol is synthesized in the skin or absorbed through the gut, it is transported to the liver where it is converted to 25-OH cholecalciferol (calcidiol) (25-hydroxycholecalciferol). This is the form that is ordinarily assayed in the blood. Calcidiol is eventually transported to the kidneys, where it is converted to 1,25-(OH)₂ cholecalciferol (calcitriol) the active form.

The compositions of the present invention can comprise from about 50 IU to about 500,000 IU, alternatively from about 500 IU to about 500,000 IU, alternatively from about 1,000 IU to about 500,000 IU of cholecalciferol, per daily dose, alternatively from about 2,000 IU to about 100,000 IU, alternatively from about 10,000 IU to about 50,000 IU, and alternatively from about 20,000 IU to about 40,000 IU, per daily dose, of cholecalciferol. With these levels of cholecalciferol administered to the human, the specific increase in blood levels of the 25-hydroxycholecalciferol in the human user will be from about 1 ng/ml to about 40 ng/ml, alternatively from about 2 ng/ml to about 30 ng/ml, alternatively from about 4 ng/ml to about 20 ng/ml, as determined by the methodology calcidiol 25-Hydroxyvitamin D ¹²⁵I RIA Kit radioimmunoassay (RIA) Catalog No./REF./KAT.-NR.:68100E manufactured, distributed and available from DiaSorin Inc., Stillwater, Minn. USA 55082.

The compositions can also comprise Vitamin D₂ (ergocalciferol). The compositions can comprise from about 50 IU to about 500,000 IU, alternatively from about 500 IU to about 500,000 IU, alternatively from about 1,000 IU to about 500,000 IU, and, alternatively from about 5,000 IU to about 500,000 IU of Vitamin D₂, per daily dose of the composition.

When present, the compositions can comprise from about 1.25 μg to about 12.5 mg, alternatively from about 12.5 μg to about 12.5 mg, alternatively from about 25 μg to about 12.5 mg, and alternatively from about 125 μg to about 12.5 mg of vitamin D₃ and/or D₂, per daily dose of the composition.

### Vitamin C

The compositions of the present invention can also comprise Vitamin C. The preferred form of Vitamin C for use in the composition is as ascorbic acid or the equivalent of a salt of ascorbic acid or the equivalent of a derivative of ascorbic acid. The vitamin C may either be in an immediate release form or a sustained release form.

Vitamin C (as calcium ascorbate) is a water-soluble compound, found in aqueous cellular compartments and is a line of defense against direct free radical exposure. Vitamin C maintains oxidative balance by effectively scavenging free radicals produced in the aqueous cellular cytoplasm and by recycling (protecting) vitamin E in cellular membranes. A preferred form of Vitamin C is as calcium ascorbate. Without being limited by theory, it is believed that ascorbate enhances the antioxidant action of vitamin E by reducing reduction of the tocopheroxyl radical. The reactions between the tocopheroxyl radical and ascorbate provide a mechanism for exporting oxidative free radicals away from the cellular membranes. In essence, tocopherols protect membranes by stopping propagation reactions of lipid peroxy radicals and ascorbate acts by protecting the membrane against possible damage from the tocopheroxyl radical. Thus, ascorbate helps to maintain oxidative balance by scavenging free radicals and recycling the useful forms of other antioxidants, such as vitamin E.

When present, the compositions can comprise from about 20 mg to about 2000 mg, alternatively from about 80 mg to about 1500 mg, and alternatively from about 100 mg to about 1000 mg of Vitamin C, per daily dose of the composition.

### Vitamin A

The compositions of the present invention can also comprise Vitamin A and/or carotene. Vitamin A and carotene can be obtained from either animal or vegetable sources. The animal form is divided between retinol and dehydroretinol whereas the vegetable carotene can be split into four very potent groups—alpha-carotene, beta-carotene, gamma-carotene and crypto-carotene. Vitamin A assists the immune system, and because of its antioxidant properties protects against disease.

Non-limiting examples of the Vitamin A useful in the present invention include vitamin A, retinol, retinyl palmitate, retinyl acetate, retinyl proprionate, beta-carotene, alpha carotene, beta-cryptoxanthin, and mixtures thereof.

When Vitamin A or one of the forms of Vitamin A is present, the compositions comprise from about 100 IU to about 10,000 IU, alternatively from about 300 IU to about 5,000 IU, alternatively from about 400 IU to about 2,000 IU, and alternatively from about 500 IU to about 1,000 IU of Vitamin A and/or form thereof, per day of the composition. The amount of Vitamin A species may be expressed as IU or as RAE (Retinol Activity Equivalent), which is equal to an equivalent amount of retinol in micrograms. For example, 10,000 IU Vitamin A is equivalent to 3000 RAE or 3000 μg retinol.

When Vitamin A (retinol) is present, the compositions can comprise from about 30 μg to about 4545 μg, alternatively from about 90 μg to about 1500 μg, alternatively from about 120 μg to about 600 μg, and alternatively from about 150 μg to about 300 μg of Vitamin A (retinol), per daily dose of the composition.

### B Vitamins

The compositions of the present invention can comprise one or more B Vitamins. The B Vitamins are water-soluble vitamins that play important roles in cell metabolism. B Vitamins are a collection of chemically distinct vitamins that often coexist in the same foods. Supplements containing eight specific B Vitamins are generally referred to as a “Vitamin B complex”. Individual B Vitamin supplements are referred to by the specific name of each vitamin (e.g. B₁, B₂, B₃, etc). The B Vitamins often work together to deliver a number of health benefits and these include, but not limited to maintenance and support of metabolic rate, maintenance of healthy skin and muscle tone, enhance immune and nervous system function, promote cell growth and division and together can also help combat the symptoms of stress, depression, and cardiovascular disease. The B Vitamin most associated with immune function is Vitamin B6 as deficiencies in this vitamin have an effect on both cellular and humoral immunity. In addition, it has been shown that B Vitamins are an essential part of energy production and can improve mood. All B Vitamins are water soluble, and are dispersed throughout the body. Most of the B Vitamins must be replenished daily, since any excess is excreted in the urine. Non-limiting examples of Vitamin B useful in the present invention include vitamin B1 (thiamin), Vitamin B2 (Riboflavin), Vitamin B3 (niacin), Vitamin B5 (pantothenic acid), Vitamin B6 (pyridoxine, pyridoxal, or pyridoxamine), Vitamin B7 (Biotin), Vitamin B9 (Folic acid), Vitamin B12 (cyanobalmin), and mixtures thereof.

When Vitamin B1 is present, the compositions can comprise from about 200 ug to about 50 mg, alternatively from about 400 μg to about 20 mg, and alternatively from about 500 μg to about 10 mg of Vitamin B1, per daily dose of the composition.

When Vitamin B2 is present, the compositions can comprise from about 100 μg to about 200 mg, alternatively from about 200 μg to about 100 mg, and alternatively from about 500 μg to about 50 mg of Vitamin B2, per daily dose of the composition.

When Vitamin B3 is present, the compositions can comprise from about 1 mg to about 500 mg, alternatively from about 2 mg to about 250 mg, and alternatively from about 5 mg to about 100 mg of Vitamin B3, per daily dose of the composition.

When Vitamin B5 is present, the compositions can comprise from about 500 μg to about 1000 mg, alternatively from about 10000 μg to about 500 mg, and alternatively from about 20000 g to about 100 mg of Vitamin B5, per daily dose of the composition.

When Vitamin B6 is present, the compositions can comprise from about 200 μg to about 500 mg, alternatively from about 500 μg to about 250 mg, and alternatively from about 11000 μg to about 100 mg of Vitamin B6, per daily dose of the composition.

When Vitamin B7 is present, the compositions can comprise from about 1 μg to about 200 μg, alternatively from about 2 μg to about 100 μg, and alternatively from about 5 μg to about 50 μg of Vitamin B7, per daily dose of the composition.

When Vitamin B9 is present, the compositions can comprise from about 50 μg to about 20000 g, alternatively from about 100 μg to about 1000 μg, and alternatively from about 200 μg to about 500 μg of Vitamin B9, per daily dose of the composition.

When Vitamin B12 is present, the compositions can comprise from about 0.5 μg to about 30000 g, alternatively from about 1 μg to about 1500 μg, and alternatively from about 2 μg to about 750 μg of Vitamin B12, per daily dose of the composition.

### Minerals, Metals, and Elements

The compositions of the present invention can include minerals, metals and elements. Non-limiting examples of minerals, metals, and elements useful in the compositions of the present invention include: iron, iodine, zinc, copper and selenium. Adequate intake of iron, zinc, copper and selenium support a Th1 cytokine-mediated immune response which helps circumvent an anti-inflammatory Th2 response and an increased risk of extracellular infections. When present, the minerals, metals and/or elements, on or in a suitable carrier, comprise from about 1% to about 50% by weight of the composition and alternatively from about 2% to about 30%, by weight of the composition.

### Plant-Derived Components

The compositions of the present invention can comprise plant-derived components. As used herein plant-derived components can include herbs including those used in traditional native American, Chinese, aruvedic and Japanese medicine, herbal extracts, and isolated active components of plants from the flower, leaves, stems, roots, and seeds of plants. Additional, non-limiting examples of plant-derived components include: Andrographis (Andrographis paniculata), borage seed oil (Borago officinalis), sage (Salvia officinalis, Salvia lavandulaefolia, Salvia lavandulifolia), Astragalus (Astragalus membraneceus), Boneset (Eupatorium perfoliatum), Chamomile (Matricaria recutita, Chamaemelum nobile), Cordyceps (Cordyceps sinensis), Echinacea (Echinacea angustifolia DC, Echinacea pallida, Echinacea purpurea), Elder (Sambucas nigra L.), Euphorbia, Garlic (Allium sativum L.), Ginsing (American ginsing, Asian ginsing, Chinese ginsing, Korean red ginsing, Panax ginsing: Panax ssp. Including P. ginsing C.C. Meyer, and P. quinquefolius L.), Goldenseal (Hydrastis canadensis L.), Greater celandine (Chelidonium majus), Horseradish (Armoracia rusticana, Cochlearia armoracia), Kiwi (Actinidia deliciosa, Actinidia chinensis), Mistletoe (Visvum album L.), Peppermint/Peppermint oil (Mentha x peperita L.), Propolis, Slippery elm (Ulmus rubra Muhl, Ulmus fulva Michx), Sorrel (Rumex acetosa L., Rumex acetosella L.), Thyme/Thymus extract (Thymus vulgaris L.), Wild indigo (Baptisia australis), and combinations and/or mixtures thereof.

### Andrographis Paniculata

The compositions of the present invention can comprise an andrographis extract, an active component thereof, or mixtures thereof. As used herein, the andrographis is a plant of the genus Andrographis, having a limited number of species within this genus largely present in Asia. Only a few of the species are medicinal. In one embodiment, the plant is of the species Andrographis paniculata, which may be referenced as Kalmegh in Ayurvedic medicine. Andrographis is typically standardized by quantifying the total amount of andrographolides, which often make up 5 to 20% of the extract.

When present, the compositions can comprise Andrographis paniculata in amounts from about 5 mg to about 50 mg, alternatively from about lOmg to about 40 mg, and alternatively from about 15 mg to about 30 mg of andrographolides, per daily dose of the composition.

### Fungal-Derived Component

The compositions of the present invention can also comprise fungal-derived components that are known to have beneficial effects with respect to enhancing immune response to respiratory conditions. Non-limiting examples of such fungal-derived components include: Maitake and Shiitake mushrooms (Grifola frondosa and Lentinus edodes respectively) and Reishi mushroom (Ganoderma lucidum); yeast (Saccharomyces cerevisiae, Saccharomyces boulardii) and cell wall extracts of yeast cells; and molds (Aspergillus). The fungal-derived components can include whole, ground, crude-sized, and superfine particle extracts, micellary extracts, and mixtures thereof, of the fungus.

A particularly useful fungal-derived component can comprise an extract of edible mushroom. Useful mushrooms are those fungi capable of forming fruit body and include but are not limited to: Lentinus edodes, Pleurotus ostreatus, Pholiota nameko, Flammulina velutipes, Tricholoma matsutake, Lyophyllum shimeji, Schizophyllum commune, Crepitodus variabilis, Lyophyllum ulmarinum, Grifola umbellate, Grifola frondosa, Coriolus versicolor, Fomes fomentarius, Volvavella volvacea, Auricularia aurcula-judae, Ganoderma lucidum, Ganoderma appanatum, Fomitopsis pinicola, Dictyophora indusiata, Sparassis crispa, Agaricus blazei, Peziza vesiculosa, and mixtures thereof. Lentinus edodes is a particularly useful non-limiting example thereof The compositions can comprise an extract, active components thereof, purified components thereof, and mixtures/combinations thereof

### Polyphenols

The compositions of the present invention can comprise at least one polyphenol. Non-limiting examples of sources of polyphenols useful in the present invention include tea extract, rosemary extract, rosemarinic acid, coffee extract, coffeic acid, turmeric extract, blueberry extract, grapeseed extract, and mixtures thereof. Polyphenols have antioxidant activity and anti-inflammatory effects.

When present, the compositions can comprise from about 0.01% to about 90%, alternatively from about 0.1% to about 35%, alternatively from about 1% to about 15%, alternatively from about 1% to about 10%, and alternatively from about 3% to about 10% of a polyphenol, by weight of the composition.

### Tea Extract

The compositions of the present invention can comprise tea extract. Tea extract contains polyphenols. Nonlimiting examples of extracts include extracts of Camellia sinensis. The tea extract has antioxidant activity so as to quench reactive oxygen species such as singlet oxygen, superoxide and hydroxyl radicals. Tea extract enhances the antioxidant defense system by preserving antioxidant enzyme activity, and can be useful to enhance immune response to a respiratory condition. Nonlimiting sources of tea extract for use in the present invention are black tea, white tea, oolong tea, and/or green tea.

When tea extract is present, the compositions can comprise from about 0.01% to about 90%, alternatively from about 0.1% to about 35%, alternatively from about 1% to about 15%, alternatively from about 1% to about 10%, and alternatively from about 3% to about 10% tea extract, by weight of the composition.

When tea extract is green tea, the compositions can comprise from about 0.01% to about 90%, alternatively from about 0.1% to about 35%, alternatively from about 1% to about 15%, alternatively from about 1% to about 10%, and alternatively from about 3% to about 10% green tea extract, by weight of the composition.

### Rosemary Extract

The compositions of the present invention can comprise rosemary extract. Rosemary extract is a polyphenol. Constituents of rosemary or rosemary extract are coffeic acid and its derivatives such as rosemarinic acid. These compounds have antioxidant activity and anti-inflammatory effects. Non-limiting sources of rosemary extract suitable for use in the present invention include rosemary.

When rosemary extract is present, the compositions can comprise from about 0.01 to about 90%, alternatively from about 0.1% to about 35% , alternatively from about 1% to about 15%, alternatively from about 1% to about 10%, and alternatively from about 3% to about 10% rosemary extract, by weight of the composition.

When rosemarinic acid is present, the compositions can comprise from about 0.01% to about 90%, alternatively from about 0.1% to about 35%, alternatively from about 1% to about 15%, alternatively from about 1% to about 10%, and alternatively from about 3% to about 10% rosemarinic acid, by weight of the composition.

### Coffee Extract

The compositions of the present invention can comprise coffee extract. Coffee extract is a polyphenol. The main constituent of coffee extract is coffeic acid and is, without being limited by theory, believed to display antioxidant activity which can be useful in enhancing immune response to a respiratory condition.

When coffee extract present, the compositions can comprise from about 0.01% to about 90%, alternatively from about 0.1% to about 35%, alternatively from about 1% to about 15%, alternatively from about 1% to about 10%, and alternatively from about 3% to about 10% coffee extract, by weight of the composition.

When coffee extract is present non-limiting sources of coffee extract include coffee bean, coffee, coffee berry, coffee fruits. When coffeic acid is present non-limiting sources of coffeic acid suitable for use in the present invention include tea, berries, coffee bean, coffee, coffee berry, coffee fruits, rosemary extract, and/or grape extract.

When coffeic acid is present, the compositions can comprise from about 0.01% to about 90%, alternatively from about 0.1% to about 35%, alternatively from about 1% to about 15%, alternatively from about 1% to about 10%, and alternatively from about 3% to about 10% coffeic acid, by weight of the composition.

### Turmeric Extract

The compositions of the present invention can comprise turmeric extract. Turmeric extract contains polyphenols. Turmeric extract is a spice which comprises a main active compound that is curcumin. Curcumin is a bioactive polyphenol plant pigment. Without being limited by theory, it is believed that curcumin has antioxidant activity and can be beneficial in enhancing immune response to a respiratory condition. A non-limiting source of turmeric extract for use in the present invention is tumeric.

When present, the compositions can comprise from about 0.01% to about 90%, alternatively from about 0.1% to about 35%, alternatively from about 1% to about 15%, alternatively from about 1% to about 10%, and alternatively from about 3% to about 10% turmeric extract, by weight of the composition.

### Blueberry Extract

The compositions of the present invention can comprise blueberry extract. Blueberry extract contains polyphenols. Blueberry extract is rich in anthocyanins which display antioxidant activity by quenching singlet oxygen. A non-limiting source of blueberry extract for use in the present invention is blueberry.

When present, the composition can comprise from about 0.01% to about 90%, alternatively from about 0.1% to about 35%, alternatively from about 1% to about 15%, alternatively from about 1% to about 10%, and alternatively from about 3% to about 10% blueberry extract, by weight of the composition.

### Grapeseed Extract

The compositions of the present invention can comprise grapeseed extract. Grapeseed extract contains polyphenols. Grape seed extract is rich in procyanidins which display antioxidant activity, which can be beneficial in enhancing immune response to a respiratory condition. Grape seed extract comprises about 38.5% procyanidins. A non-limiting source of grapeseed extract for use in the present invention is grape seed.

When present, the compositions can comprise from about 0.01% to about 90%, alternatively from about 0.1% to about 35%, alternatively from about 1% to about 15%, alternatively from about 1% to about 10%, and alternatively from about 3% to about 10% grapeseed extract, by weight of the composition.

### Carotenoids

The composition of the present invention can comprise a carotenoid. A “carotenoid” is a class of pigments occurring in the tissues of higher plants, algae, bacteria and fungi. They are usually yellow to deep red crystalline solids, soluble in fats and oils, insoluble in water, high-melting, stable to alkali, unstable to acids and oxidizing agents, their color is easily destroyed by hydrogenation or by oxidation, and some are optically active. Carotenoids are natural pigments synthesized by plants and microorganisms that are thought to function as light absorbing pigments during photosynthesis and to protect cells from photosensitization. Structurally, carotenoids consist of eight isoprenoid units joined so that their arrangement is reversed at the center of the molecule. Carotenoid structure strongly affects the physical properties, chemical reactivity and biologic functions of these compounds. It has been suggested that the size, shape, hydrophobicity and polarity of individual carotenoids may dramatically affect the bioavailability, absorption, circulation, tissue and subcellular distribution and excretion in mammals. Carotenoids have demonstrated antioxidant activity and as well as other biological activities in addition to maintaining oxidative balance.

When a carotenoid is present, the carotenoid is selected from the group consisting of lutein, astaxanthin, zeaxanthin, bixin, lycopene, and mixtures thereof.

### Lutein

The compositions of the present invention can comprise lutein. Lutein is a powerful antioxidant. Lutein and zeaxanthin are structural isomers of one another. Lutein can be extracted in crystalline form from marigolds. Dietary sources of lutein include mustard greens, spinach, kale, broccoli, leaf lettuce, green peas, brussel sprouts, corn, some squash and green beans.

When present, the compositions can comprise at least about 0.01%, alternatively from about 0.01% to about 20%, and alternatively from about 0.05% to about 10% lutein, by weight of the composition.

Zeaxanthin

The compositions of the present invention can comprise zeaxanthin. Zeaxanthin is also a powerful antioxidant. Zeaxanthin can be extracted in crystalline form from marigolds. Dietary sources of zeaxanthin include mustard greens, spinach, kale, broccoli, leaf lettuce, green peas, brussel sprouts, corn, some squash and green beans.

When present, the compositions can comprise at least about 0.01%, alternatively from about 0.01% to 20%, and alternatively from about 0.05% to about 10% zeaxanthin, by weight of the composition.

### Astaxanthin

The compositions of the present invention can comprise astaxanthin. Astaxanthin is also a powerful antioxidant and can be provided as free astaxanthin or as astaxanthin diester. Naturally produced astaxanthin can be obtained from fungi, crustaceans, and algae, e.g., Haematococcus sp. Astaxanthin is also produced by wild-type and genetically engineered Pfaffia yeast, and is commercially available from Archer Daniels Midland Co.; Aquasearch Inc.; AstaCarotene AB; Cyanotech Corporation and Micro Gaia, Inc. Synthetically produced astaxanthin is also commercially available from Hoffman-LaRoche, Ltd.

When present, the compositions can comprise at least about 0.01%, alternatively from about 0.01% to about 20%, and alternatively from about 0.05% to about 10% astaxanthin, by weight of the composition.

### Bixin

The compositions of the present invention can comprise bixin. Bixin is a naturally occurring carotenoid, found in the pulp of the B. orellana seed (also called annatto seed), used all over the world as a red-orange dye for coloring rice, cheeses, soft drinks, oil, butter, soup and cosmetics. As annatto extract, it is used as a color additive in food. Bixin is an antioxidant that can scavenge free radicals and prevent oxidative damage.

When present, the compositions can comprise at least about 0.01%, alternatively from about 0.01% to about 20%, and alternatively from about 0.05% to about 10% bixin, by weight of the composition.

### Lycopene

The compositions of the present invention can comprise lycopene. Lycopene is an open-chain unsaturated carotenoid that imparts red color to tomatoes, guava, rosehip, watermelon and pink grapefruit. Lycopene is a proven antioxidant, which neutralize free radicals that may damage the body's cells.

When present, the compositions can comprise at least about 0.01%, alternatively from about 0.01% to about 20%, and alternatively from about 0.05% to about 10%, lycopene, by weight of the composition.

### Antioxidants

The composition of the present invention can comprise an antioxidant in addition to the vitamins, plant-derived components, elements, and carotenoids described above as having antioxidant properties. An antioxidant is an enzyme or other organic molecule that can counteract the damaging effects of oxygen in tissues. Although the term technically applies to molecules reacting with oxygen, it is often applied to molecules that protect from any free radical. Antioxidants can include natural and synthetic vitamins, plant-derived components and carotenoids, many of which have been described above, in addition to the antioxidants described in this section.

When an antioxidant is present, non-limiting examples of such antioxidants include tocopherols (Vitamin E), Vitamin C (described above), Vitamin A (described above), CoQ10, plant-derived materials (described above), carotenoids (described above), selenium (described above), and mixtures thereof.

### Vitamin E

The compositions of the present invention can comprise Vitamin E. Vitamin E is a lipid soluble compound and the most significant antioxidant activity of vitamin E is localized to cellular membranes. Vitamin E maintains oxidative balance by protecting cellular membranes from lipid peroxidation. Vitamin E is a lipid soluble antioxidant and provides defenses against cellular oxidative damage. Major dietary sources of vitamin E are vegetable oils, margarine and shortening, with nuts, seeds, whole grains and wheat germ providing additional sources. The term “Vitamin E” typically includes eight different chemical forms: four tocopherols and four tocotrienols. The most biologically active form of vitamin E is alpha-tocopherol.

When present, the compositions can comprise from about 1 mg to about 1000 mg, alternatively from about lmg to about 800 mg, and alternatively from about 2 mg to about 200 mg of vitamin E, per daily dose of the composition.

When present, the compositions can comprise at least about 0.01%, alternatively from about 0.01% to about 10%, and alternatively from about 0.2% to about 5% Vitamin E, by weight of the composition.

### Coenzyme Q10

The compositions of the present invention can comprise coenzyme Q10 (CoQ10). Coenzyme Q10 is a powerful naturally occurring compound that promotes chemical reactions and aids in protecting mammals from free radicals. It is also called ubiquinone. Coenzyme Q10 (CoQ10) is naturally present in foods, and can be synthesized by mammals from the amino acid tyrosine during a multistage (17 stages) process requiring eight vitamins and several trace elements. Coenzyme Q10 provides antioxidant qualities as well as the control it exercises on the flow of oxygen within cells, assistance with cardiovascular functioning, the production of energy, assistance with absorption of other nutrients as well as having immune boosting properties.

When present, the compositions can comprise at least about 0.01%, alternatively from about 0.01% to about 10%, and alternatively from about 0.2% to about 5% Coenzyme Q10, by weight of the composition.

When present, the compositions can comprise from about lmg to about 400 mg, alternatively from about 2 mg to about 400 mg, and alternatively from about 3 mg to about 300 mg of Coenzyme Q10, per daily dose of the composition.

### Method of Manufacture

The compositions and various components used in the present invention may be manufactured in accordance with known methods.

### EXAMPLES

The following examples further describe and demonstrate embodiments within the scope of the invention. The examples are given solely for the purpose of illustration and are not to be construed as limitations of the present invention, as many variations thereof are possible without departing from the spirit and scope of the invention.

### Example I

A 60 kg woman is diagnosed with irritable bowel syndrome and the colonoscopy demonstrates inflammation around the nerves in the wall of the colon which control peristalsis. The fecal surrogate markers of inflammation are also raised. She is prescribed a pharmaceutical composition comprising 2400 milligrams delayed-release mesalamine, to be taken once-daily. A repeat biopsy of the colonic wall will be taken 6 months later to determine if there is a decrease in the inflammation around the nerves which control peristalsis.

### Example II

A 65 kg woman is diagnosed with irritable bowel syndrome and the colonoscopy demonstrates inflammation around the nerves in the wall of the colon which control peristalsis. The fecal surrogate markers of inflammation are also raised. She is prescribed a pharmaceutical composition comprising 4800 milligrams delayed-release mesalamine, to be taken on alternate days. A repeat biopsy of the colonic wall will be taken 6 months later to determine if there is a decrease in the inflammation around the nerves which control peristalsis.

### Example III

A 75 kg man has been using numerous OTC medicines multiple times per day for what his general physician calls a “spastic colon” (pm antispasmodics, anti-diarrheals, laxatives and analgesics). A colonoscopy shows inflammation in the mucosa and wall of the colon. There is also a raised level of inflammatory surrogate markers in the fecal effluent specimens. The patient is prescribed a pharmaceutical composition to be taken once-daily. Each unit dose of the pharmaceutical composition comprises 4800 milligrams of delayed-release mesalamine. The patient takes a unit dose of the pharmaceutical composition once per day every morning. A follow-up colonic biopsy will be taken 6 months later and determine if there is a decrease in the degree of inflammation. A repeat laboratory evaluation of fecal inflammatory markers will also be evaluated to see if it also shows a reduction at 6 months.

### Example IV

A 75 kg man is diagnosed with inflammatory irritable bowel syndrome by colonoscopy which demonstrates inflammation in the wall of his colon. The patient is prescribed a pharmaceutical composition to be taken twice each week. Each unit dose of the pharmaceutical composition comprises 7200 milligrams of delayed-release mesalamine. The amount of delayed-release mesalamine absorbed into the mucosa and colonic wall (mg/gram of tissue) proved by follow-up biopsies taken is the equivalent to that of 2400 milligrams mesalamine taken once-daily.

### Example V

A 67 kg woman is diagnosed with inflammatory irritable bowel syndrome by colonoscopy which demonstrates inflammation in the wall of the colon. The patient is prescribed a pharmaceutical composition to be taken once daily and a probiotic, also to be taken once-daily. Each unit dose of the pharmaceutical composition comprises 2400 milligrams of delayed-release mesalamine. Each unit dose of probiotic to be taken once daily comprises 10⁸ CFU Bifidobacterium infantis 35624.

### Example VI

A 78 kg man is diagnosed with inflammatory irritable bowel syndrome by colonoscopy which demonstrates inflammation in the wall of the colon. The patient is prescribed a pharmaceutical composition to be taken once daily and a probiotic, also to be taken once-daily. Each unit dose of the pharmaceutical composition comprises 4800 milligrams of delayed-release mesalamine. Each unit dose of probiotic to be taken once daily comprises 10¹⁰ CFU Bifidobacterium infantis 35624. A repeat biopsy of the colonic wall will be taken 6 months later to determine if there is a decrease of the inflammation around the nerves which control peristatlsis.

### Example VII

A 85 kg man is diagnosed with inflammatory irritable bowel syndrome by colonoscopy which demonstrates inflammation in the wall of the colon. The patient is prescribed a pharmaceutical composition to be taken on alternate days and a probiotic, also to be taken on an alternate day dosing regimen. Each unit dose of the pharmaceutical composition comprises 2400 grams of delayed-release mesalamine. Each unit dose of probiotic to be taken on alternate days comprises 10⁸ CFU Bifidobacterium infantis 35624.

### Example VIII

A 76 kg woman is diagnosed with inflammatory irritable bowel syndrome by colonoscopy which demonstrates inflammation in the wall of the colon. The patient is prescribed a pharmaceutical composition to be taken on alternate days and a probiotic, also to be taken on an alternate day dosing regimen. Each unit dose of the pharmaceutical composition comprises 2400 milligrams of delayed-release mesalamine. Each unit dose of probiotic to be taken on alternate days comprises 10⁵ CFU Bifidobacterium infantis 35624.

### Example IX

A 67 kg woman is diagnosed with inflammatory irritable bowel syndrome by colonoscopy which demonstrates inflammation in the wall of the colon. The patient is prescribed a pharmaceutical composition to be taken on alternate days and a probiotic, also to be taken on an alternate day dosing regimen. Each unit dose of the pharmaceutical composition comprises 4800 milligrams of delayed-release mesalamine. Each unit dose of probiotic to be taken on alternate days comprises 10⁵ CFU Bifidobacterium infantis 35624.

### Example X

A 72 kg woman is diagnosed with inflammatory irritable bowel syndrome by colonoscopy which demonstrates inflammation in the wall of the colon. The patient is prescribed a pharmaceutical composition to be taken on alternate days and a probiotic, also to be taken on an alternate day dosing regimen. Each unit dose of the pharmaceutical composition comprises 4800 milligrams of delayed-release mesalamine. Each unit dose of probiotic to be taken on alternate days comprises 10¹⁰ CFU Bifidobacterium infantis 35624.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.
¹Gionchetti P, Amadini C, Rizzello F, Venturi A, Palmonari V, Morselli C, et al. Probiotics—role in inflammatory bowel disease. Dig Liver Dis. 2002;34(Suppl 2):S58-62.
²Floch M H, Fuchs H M. Modification of stool content by increased bran intake. Am J ClinNutr. 1978;31(10 Suppl):S185-9.

What is claimed is:

1. A method of treating an inflammatory bowel disorder; comprising administering to a mammal in need of such treatment a composition comprising at least one active agent; and a probiotic.

2. The method of claim 1, wherein said active agent is selected from the group comprising: aminosalicylate, sulfasalazine, 5-aminosalicylic acid, 4-aminosalicylic acid, benzalazine, dihydrochloride salt, olsalazine, balsalazide, bismuth subsalicylate, and mixtures thereof.

3. The method of claim 1, wherein said composition comprises from about 1 mg to about 8000 mg of said active agent.

4. The method of claim 1, wherein said composition comprises from about 200 mg to about 4800 mg of said active agent.

5. The method of claim 1, wherein said probiotic is a Bifidobacterium sp.

6. The method of claim 5, wherein said Bifidobacterium sp. comprises bacteria selected from the group consisting of Bifidobacterium longum, Bifidobacterium infantis, Bifidobacterium bifidum, and mixtures thereof.

7. The method of claim 5, wherein the Bifidobacterium sp. comprises Bifidobacterium infantis.

8. The method of claim 5 wherein a single dose of the probiotic comprises at least about 10⁵ cfu of the Bifidobacterium sp.

9. The method according to claim 8 wherein a single dose of the probiotic comprises from about 10⁸ cfu to about 10¹² cfu of the Bifidobacterium sp.

10. The method of claim 1, wherein said inflammatory bowel disorder is selected from the group consisting of inflammatory irritable bowel syndrome, irritable bowel syndrome-diarrhea, irritable bowel syndrome-constipation, irritable bowel syndrome-mixed, irritable bowel syndrome-alternating, dyspepsia, microscopic colitis, lymphocytic colitis, collagenous colitis, indeterminant colitis, celiac disease, and combinations thereof.

11. The method of claim 1, wherein said inflammatory bowel disorder is selected from the group consisting of inflammatory irritable bowel syndrome, irritable bowel syndrome-diarrhea, irritable bowel syndrome-constipation, irritable bowel syndrome-mixed, irritable bowel syndrome-alternating, and combinations thereof.

12. The method of claim 1, wherein said inflammatory bowel disorder is inflammatory irritable bowel syndrome.

13. The method of claim 1, wherein administration of the composition is selected from the group consisting of oral, rectal, intramuscular, subcutaneous, intravenous, transdermal, and combinations thereof.

14. The method of claim 13, wherein administration of the composition is selected from the group consisting of oral, rectal, and combinations thereof.

15. The method of claim 13, wherein administration of the composition is oral.

16. The method of claim 13, wherein administration of the composition is at least once daily.

17. The method of claim 5, wherein administration of the Bifidobacterium sp. and said active agent is contemporaneous.

18. The method of claim 5, wherein the composition comprising said active agent also comprises the Bifidobacterium sp.

19. The method of claim 5, wherein administration of the active agent is prior to the administration of the Bifidobacterium sp.

20. The method of claim 5, administration of the Bifidobacterium sp. is prior to the administration of the active agent.

21. The method of claim 1, further comprising administering to the mammal an antibiotic.

22. The method of claim 21, wherein the antibiotic is selected from the group consisting of metronidazole, cephalexin, ciprofloxacin, levofloxacin, moxifloxacin, gemifloxacin, balofloxacin, gatifloxacin, grepafloxacin, pazufloxacin, sparfloxacin, temafloxacin, enoxacin, fleroxacin, lomefloxacin, nadifloxacin, norfloxacin, ofloxacin, pefloxacin, rufloxacin, trovofloxacin, tosufloxacin, clindamycin, tetracycline, chloramphenicol, cefoxitin, cefmetazole, cefotetan, doxycycline, erythromycin, imipenem, meropenem, ticarcillin, pipercillin, mezocillin, tazobactam, ampicillin, and combinations thereof.

23. The method of claim 22, wherein administration of the antibiotic is prior to the administration of the Bifidobacterium sp. and prior to the administration of the active agent.

24. The Method of claim 1, wherein said composition further comprising an additional component selected from the group consisting of an additional probiotic strain of bacteria; prebiotics; fiber; vitamins; minerals; metals; elements; plant-derived components; fungal-derived components; carotenoids; anti-oxidants and combinations thereof.

25. A method of treating an inflammatory bowel disorder; comprising administering to a mammal in need of such treatment a probiotic, an active agent, and an antibiotic.

26. The method of claim 25, wherein the administration of the antibiotic is prior to the administration of the probiotic and prior to the administration of the active agent.

27. The method of claim 25, comprising administration of a composition, wherein the composition comprises the probiotic and the active agent.

28. The method of claim 25, wherein said active agent is selected from the group comprising: aminosalicylate, sulfasalazine, 5-aminosalicylic acid, 4-aminosalicylic acid, benzalazine, dihydrochloride salt, olsalazine, balsalazide, bismuth subsalicylate, and mixtures thereof.

29. The method of claim 28, wherein said active agent is a 5-aminosalicylic acid.

30. The method according to claim 25, wherein said antibiotic is selected from the group consisting of metronidazole, cephalexin, ciprofloxacin, levofloxacin, moxifloxacin, gemifloxacin, balofloxacin, gatifloxacin, grepafloxacin, pazufloxacin, sparfloxacin, temafloxacin, enoxacin, fleroxacin, lomefloxacin, nadifloxacin, norfloxacin, ofloxacin, pefloxacin, rufloxacin, trovofloxacin, tosufloxacin, clindamycin, tetracycline, chloramphenicol, cefoxitin, cefmetazole, cefotetan, doxycycline, erythromycin, imipenem, meropenem, ticarcillin, pipercillin, mezocillin, tazobactam, ampicillin, and combinations thereof.

31. The Method of claim 25, further comprising a an additional component selected from the group consisting of an additional probiotic strain of bacteria; prebiotics; fiber; vitamins; minerals; metals; elements; plant-derived components; fungal-derived components; carotenoids; anti-oxidants and combinations thereof.

32. A kit comprising:
a) a probiotic; and
b) an active agent.

33. The kit of claim 32, wherein said active agent wherein said active agent is selected from the group comprising: aminosalicylate, sulfasalazine, 5-aminosalicylic acid, 4-aminosalicclic acid, benzalazine, dihydrochloride salt, olsalazine, balsalazide, bismuth subsalicylate, and mixtures thereof.

34. The kit of claim 33, wherein the active agent is a 5-aminosalicylic acid.

35. The kit of claim 32, wherein said probiotic is a Bifidobacterium sp.

36. The kit of claim 32, comprising a first composition, wherein the first composition comprises from about 1 mg to about 8000 mg of said 5-aminosalicylic acid.

37. The kit of claim 36, comprising a second composition, wherein the second composition comprises said Bifidobacterium sp.

38. The kit of claim 37, wherein said Bifidobacterium sp. comprises bacteria selected from the group consisting of Bifidobacterium longum, Bifidobacterium infantis, Bifidobacterium bifidum, and mixtures thereof.

39. The kit of claim 37, wherein the second composition comprises at least about 10⁵ cfu of the Bifidobacterium sp.

40. The kit of claim 39, wherein the second composition comprises from about 10⁸ cfu to about cfu 10¹² of the Bifidobacterium sp.

41. The kit of claim 38, wherein the Bifidobacterium sp. is Bifidobacterium infantis.

42. The kit of claim 38, wherein the Bifidobacterium sp. is Bifidobacterium infantis.

43. The kit of claim 32, further comprising an antibiotic.

44. The kit of claim 43, wherein said antibiotic is selected from the group consisting of metronidazole, cephalexin, ciprofloxacin, levofloxacin, moxifloxacin, gemifloxacin, balofloxacin, gatifloxacin, grepafloxacin, pazufloxacin, sparfloxacin, temafloxacin, enoxacin, fleroxacin, lomefloxacin, nadifloxacin, norfloxacin, ofloxacin, pefloxacin, rufloxacin, trovofloxacin, tosufloxacin, clindamycin, tetracycline, chloramphenicol, cefoxitin, cefmetazole, cefotetan, doxycycline, erythromycin, imipenem, meropenem, ticarcillin, pipercillin, mezocillin, tazobactam, ampicillin, and combinations thereof.

45. The Kit of claim 32, further comprising a an additional component selected from the group consisting of an additional probiotic strain of bacteria; prebiotics; fiber; vitamins; minerals; metals; elements; plant-derived components; fungal-derived components; carotenoids; anti-oxidants and combinations thereof.
